(19) United States
(12) Patent Application Publication
(10) Pub. No.: US 2021/0385621 A1
(43) Pub. Date: Dec. 9, 2021

(54) Method and System for Mapping Persons and Resources

(71) Applicant: WHERE.PLACE, Anaheim, CA (US)

(72) Inventors: Hoang Doan Hong Vo, Anaheim, CA (US); Ronald B. Koo, Los Altos, CA (US); Dennis Chang, Seal Beach, CA (US)

(73) Assignee: WHERE.PLACE, Anaheim, CA (US)

(21) Appl. No.: 17/302,881

(22) Filed: May 14, 2021

Related U.S. Application Data
(60) Provisional application No. 63/035,121, filed on Jun. 5, 2020.

(51) Int. Cl. H04W 4/029; H04W 4/80

(52) U.S. Cl. CPC H04W 4/029; G16H 50/80; H04W 4/80

(57) ABSTRACT

Methods and systems are provided for mapping persons or resources within an environment. One application of the methods and systems provided is contact tracing.

### CROSS REFERENCE TO RELATED APPLICATIONS

This non-provisional application claims the benefit of priority to U.S. Provisional Patent Ser. No. 63/035,121, filed Jun. 5, 2020, the entire contents of which are hereby incorporated by reference in their entirety.

### TECHNICAL FIELD

The present invention relates to mapping people and resources in an environment.

### BACKGROUND

There are many applications and uses for a system of mapping people and/or resources (such as equipment) within an environment. One example is tracking the locations of employees or other people within a secure facility to ascertain which areas such persons have accessed during the course of the day. Another example is tracking the locations of patients and staff at an assisted living facility to ensure that staff have attended to patients and otherwise performed their duties. Another application would be to determine whether two or more individuals have been in close proximity with each other; this could be useful for helping law enforcement ascertain whether a suspected criminal or terrorist has been in contact with other individuals. Yet another application is tracking the location of tagged equipment in a hospital. Such methods and systems can also be used to track the location of non-human animals, such as in a wildlife preserve, zoo or aquarium. A very important application of the claimed methods and systems of the invention is contact tracing.

Contact tracing is a method of finding the people who have been exposed to a person with a highly communicable disease, such as the disease caused by severe acute respiratory syndrome coronavirus 2 (SARS-CoV-2), aka COVID-19, by back-tracing the steps of a patient with the disease to determine every individual who might have come in contact with such patient over a certain time period. Traditionally, the contact tracing process is very laborious. See, e.g., K. Landman, “How the Painstaking Work of Contact Tracing Can Slow the Spread of an Outbreak,” Mar. 10, 2020, https://www.npr.org/sections/health-shots/2020/03/10/814129534/how-the-painstaking-work-of-contact-tracing-can-slow-the-spread-of-an-outbreak (retrieved Apr. 17, 2020). During this process, public health workers talk to the patients with communicable diseases. Knowing how such a disease will spread, the public health workers call other likely people who were in contact with the infected individual and therefore might have been infected to inform them of the potential exposure, ask their health status, and request them to take appropriate action, such as quarantine.

The number of potentially infected people can increase very quickly with a disease that has a high reproduction rate. The sheer numbers could overwhelm the ability of public health workers to keep up. With the advent of mobile devices (such as cell phones) that users often carry with them for a large fraction of the day, many proposals have been put forth to perform automated contact tracing using mobile devices. See, e.g., such as U.S. Pat. Nos. 7,705,723 and 8,049,614. As a result, using automated, digital contact tracing, public health workers would be able to contact trace a far greater number of people than previously. Such a process utilizing automated, digital contact tracing would be more reliable than relying on the memories of the infected patient.

Utilizing digital contact tracing, there are essentially three major methods of determining whether people are in contact with each other. The first method relies upon the relative positioning of mobile devices to one another when they are within a short distance. For example, if smart phones are equipped with Bluetooth radios that have been turned on, and when the smart phones are within approximately three meters of each other, the smart phones can store the identifiers of all the smart phones that are in their vicinity. See, e.g., U.S. Pat. Nos. 8,645,538 and 8,405,503; see also, e.g., O. Seiskari, “Why use Bluetooth for contact tracing?”, https://medium.com/indooratlas/why-use-bluetooth-for-contact-tracing-1585feb024dc (retrieved Apr. 17, 2020); NextTrace, “Participatory Digital Contact Tracing Through User Interviews and On-ramp to Testing,” https://docs.google.com/d/-linQyAzC8eibq2kCz487Xb7dkjNaWtM3uzL_ceNrgpXI/edit (retrieved Apr. 17, 2020); and Government Technology Agency of Singapore “Help fight the spread of COVID-19 with TraceTogether!”, https://www.youtube.com/watch?time_continue=97&v=buj8ZTRtJes&feature=emb_title (retrieved Apr. 17, 2020). A variant of this approach uses RFID tags and RFID readers for indoor location recognition; one disadvantage of this approach is that it requires additional, typically expensive equipment, given that most mobile devices do not read or broadcast RFID signals.

The second method relies upon the absolute positioning of mobile devices with respect to radio beacons such as those of the Global Positioning System (GPS), Wi-Fi, or both. With GPS, the mobile devices can calculate their longitude and latitude on the planet earth. With Wi-Fi, the mobile devices can determine that they are within range of a specific Wi-Fi access point. The mobile devices are deemed to have come within contact of each other if the longitude and latitude are approximately the same, or if the same access point is within range. See, e.g., Raskar et al., “Apps Gone Rogue: Maintaining Personal Privacy in an Epidemic,” Mar. 20, 2020, https://arxiv.org/abs/2003.08567 (accessed Apr. 16, 2020); and EPIWORK, “Developing the Framework for an Epidemic Forecast Infrastructure,” Nov. 15, 2011, https://cordis.europa.eu/docs/projects/cnect/7/231807/080/deliverables/001-D56.pdf (accessed Apr. 17, 2020) (both describing the use of absolute positioning techniques). Notably, using such methods, the relative positions of two mobile devices is not actually calculated to determine whether the two mobile devices are within range of each other. Rather, the two mobile devices are deemed to be within range of each other if they both are within range of a specific Wi-Fi access point or beacon. Obviously, there are many situations where two devices could be within range of a common Wi-Fi access point or beacon but not within range of each other; conversely, two devices could be within range of each other without being within range of the same Wi-Fi access point or beacon.

The third method is a combination of both the first and the second methods together.

All three methods suffer from a key drawback in that they are unable to distinguish between people and objects located on different floors or altitudes. The above-described methods fundamentally assume that the people carrying the mobile devices exist in a two-dimensional world, and hence, the people are not on separate floors of a building or at different altitudes. One commentator noted: “Anyway, could the GPS-that-is-actually-WiFi be used for contact tracing? The biggest issue seems to be altitude information, which is not consistently provided by the built-in location services (apparently Google's WiFi world is 2-dimensional) so people on different floors of the same building could be incorrectly detected as being close to each other.” O. Seiskari, “Why use Bluetooth for contact tracing?”, https://medium.com/indooratlas/why-use-bluetooth-for-contact-tracing-1585feb024dc (retrieved Apr. 17, 2020). Without the ability to distinguish on which floor of a building people are, mapping people to a specific room in the building is impossible.

While the methods and systems of the invention can be applied to outdoor and indoor environments, as well as environments comprising both indoor and outdoor components, the main advantages of the invention accrue to indoor environments. Notably, indoor environments are of the greatest significance in the context of contact tracing. Professor Erin Bromage, Associate Professor of Biology at the University of Massachusetts Dartmouth, noted: “Importantly, of the countries performing contact tracing properly, only a single outbreak has been reported from an outdoor environment (less than 0.3% of traced infections). Indoor spaces, with limited air exchange or recycled air and lots of people, are concerning from a [COVID-19] transmission standpoint.” E. Bromage, “The Risks—Know Them—Avoid Them,” https://www.erinbromage.com/post/the-risks-know-them-avoid-them (retrieved Apr. 17, 2020). Lacking reference to floors, rooms, and hallways, the three methods above might produce erroneous results in which people are considered to have contact even though they are separated by walls or floors. What is needed therefore are methods and systems that can ascertain whether people have been on the same floor and in the same room.

Another drawback of the prior art is that they could not effectively handle environments consisting of a mix of indoor and outdoor spaces. For example, if a person exits a building to a courtyard or outdoor terrace, the person's location could not be determined accurately. Similarly, on a corporate campus with multiple, separate buildings, the location of a person walking between buildings could not be determined accurately. As a result, the location trail information would be incomplete, rendering any contact tracing inaccurate and prone to erroneous conclusions. What is needed therefore are systems and methods of mapping and contact tracing that can accurately handle mixed indoor/outdoor environments.

As noted above, many of the prior art approaches rely entirely on the relative positions of two mobile devices or the relative positions of mobile devices to one or more beacons. In other words, these approaches rely on two devices being within proximity of each other at the same time. These approaches do not use maps of the environment. Consequently, they do not take into account the presence of walls, floors or other physical barriers that can eliminate or reduce the risk of infection. Moreover, such approaches do not take into account that infection can be spread through contact with surfaces that an infected person has touched or by breathing in air containing infectious agents left by an infected person who was previously in the room. Therefore, such approaches would erroneously conclude that a person has not been exposed just because that person was not in proximity to an infected person at the same time.

To the extent that prior art approaches incorporated the use of maps, they only used static maps, such as architectural floor plans. Such maps typically do not include accurate dimensions and accurate GPS coordinates of physical features and structures, such as doors, windows, air ducts and Wi-Fi access points. The lack of such information makes it impossible to perform sophisticated contact tracing analysis. What is needed therefore are systems and methods that utilize digital interactive maps that allow for more accurate location trails and more sophisticated contact tracing analysis.

### SUMMARY OF THE INVENTION

In accordance with one embodiment of the invention, there is provided a method and system for contact tracing that maps users inside of buildings, that compares the location trails of mobile devices, that localizes users by analyzing the measurements on the signals of the wireless communication devices inside of a building with reference to a map, and that determines whether people have been on the same floor, the same room, or the same hallway. While the term “contact tracing” is commonly used to refer to tracing an individual's contacts during a period of time to determine whether an individual or individuals have been exposed to an infectious disease, as used herein, the term “contact tracing” is intended to have the broader meaning of tracing an individual's contacts for any reason. In some cases, mapping certain areas of a building to the “same” floor can present logistical challenges, given that some areas may be between floors, such as stairs, escalators or landings between floors.

In such cases, these areas can be assigned to the closest floor based upon a set of rules for such spaces. Similar challenges can exist in determining whether people are in the “same” room, as some spaces may be partially connected or connected at certain times but not at other times. Examples include meeting rooms that have movable dividers that can split a room into multiple, sealed-off rooms or rooms with high dividers that obstruct movement and partially obstruct airflow. Similarly, elevators can be connected to multiple floors and multiple rooms. To properly map such spaces, it is necessary to develop a set of rules for assigning such spaces to a specific floor and specific room. Persons can be considered to be considered to be on “approximately the same floor” or in “approximately the same room” if they are in a space (e.g., stairwell, landing) that is contiguous to such floor or room.

To determine whether two individuals have been in the same location at the same time requires that one track not just location but also an associated time. Location trails are sequences of locations that are tracked at uniform time intervals. Alternatively, if location information is not recorded at regular time intervals, each location must be stored in the location along with a time indicating when a person or that person's mobile device was at such location. Given that time information may not be precise (i.e., error in measurement), it must be recognized that intersecting location trails do not necessarily mean that two individuals were in proximity of each other. In the context of the present application, the term “approximately the same time” shall mean within fifteen minutes apart.

In another embodiment of the invention, there is presented a mapping system comprising: a first mobile device that is associated with a first person; a wireless communication device which can communicate to the first mobile device, wherein the communication device sends identifying information about the communication device to the first mobile device and wherein the first mobile device receives measurements on signals received from the wireless communication device; a map stored on the first mobile device, wherein the map is marked with location of the wireless communication device; whereby a first location trail is calculated based upon the map, the identifying information and the measurements, and wherein the location trail is stored on the first mobile device; a second mobile device associated with a second person, wherein the second mobile device receives the first location trail and compares the first location trail with a second location trail associated with the second person in order to determine whether the first mobile device has been on the same building floor at approximately the same time as the second mobile device. In yet another embodiment, the second mobile device determines if the first mobile device has been in the same room at approximately the same time as the second mobile device.

In yet another embodiment, there is presented a system for mapping the location of a mobile device associated with a person within an environment comprising: a map server which stores a map of the environment that includes the location of wireless communication devices located within the environment; wherein the mobile device obtains data needed to calculate time-bound location trails and send the data to the map server; wherein the map server determines the mobile device's location based upon the data and the map, and stores the location in a location trail stored in a database, wherein the location trail is associated with the mobile device. In another embodiment, the map server also compares the location trail associated with the mobile device with other location trails associated with other mobile devices. One potential purpose of such a comparison is to perform contact tracing. Another potential purpose is for law enforcement to determine whether a suspected criminal or suspected terrorist has been in communication with another individual.

In yet another embodiment, there is presented a distributed system of mapping the location of a mobile device associated with a person within an environment comprising: a map server which stores a map of the environment that includes the location of wireless communication devices located within the environment; and a first database stored on the map server, wherein the first database stores maps and wireless footprint data; a second database stored on the mobile device, wherein the second database stores maps and wireless footprint data retrieved from the map server; a third database stored on the mobile device, wherein the third database stores a location trail of the mobile device, wherein the mobile device obtains data needed to calculate its location; wherein the mobile device receives the map from the map server upon providing the data to the map server; wherein the mobile device calculates the location trail based upon the data, the footprint data and the map, and stores the location trail in the third database. In certain embodiments, the second and third databases can be combined into a single database. Likewise, the first database can be split into multiple databases.

In the above-described embodiments, as well as various other embodiments, the map server can be located on-site or off-site. Data can be transmitted to or from the map server using various well-known methods, including transmission through the internet. Preferably, the data is encrypted before transmission. In certain embodiments, any databases, whether stored on the mobile device or on a remote server, are also encrypted.

Also presented are systems of contact tracing a person associated with a mobile device comprising: an infected repository server that receives a location trail associated with the mobile device calculates an infectivity window of when an infected person can transmit a specific disease to another person; and a database that stores the location trail bounded by the infectivity window; wherein the location trail bounded by the infectivity window can be sent from the infected repository server to other mobile devices associated with other people; wherein such other mobile devices compare the location trail bounded by the infectivity window to other location trails stored on the other mobile devices, and determine whether the other people have been exposed to the disease based upon the comparison.

Also presented are systems of contact tracing a person associated with a mobile device comprising: a repository server that receives a location trail associated with the mobile device calculates a meeting window of when said person said person has met or been in contact with another person; and a database that stores the location trail bounded by the meeting window;

Atty Dkt No. 30010-0001US wherein the location trail bounded by the meeting window can be sent from the repository server to other mobile devices associated with other people; wherein such other mobile devices compare the location trail bounded by the meeting window to other location trails stored on the other mobile devices, and determine whether said person has met or been in contact with other people based upon the comparison.

In another embodiment, there is presented a system of contact tracing a person associated with a mobile device, wherein the person is infected or potentially infected with a specific disease, comprising: an infected repository server that receives a location trail associated with the mobile device and computes an infectivity window of when an infected person can transmit the specific disease to another person; a database that stores the location trail bounded by the infectivity window; wherein a device associated with a person other than the infected or potentially infected person can request and receive the location trail bounded by the infectivity window; wherein the requesting device can compare the location trail bounded by the infectivity window to another location trail stored on the requesting device, and determine whether the other person has been exposed to the disease based upon the comparison.

In the above-described embodiments, as well as other embodiments, the infected repository server or repository server can be located on-site or off-site. Mobile devices associated with a person, such as an infected or potentially infected person, can transmit location trail information and other information relating to the infection or potential infection to the infected repository server or repository. Data can be transmitted to or from the infected repository server or repository server using various well-known methods, including over the internet. Location trails can be calculated based in part on propagation delays of wireless signals from wireless communication devices located within the vicinity of the mobile device. In certain embodiments, the propagation delays are estimated through modeling of the paths from the map server to the wireless communication device and from the wireless communication device to the mobile device. In certain contact-tracing embodiments, estimates of the likelihood of infection can be improved using (i) models of time decay of airborne virus particles and/or (ii) models of the airflow in a room or between rooms.

Also presented are various methods for mapping the location of a first person within an environment. In one embodiment, the method comprises: providing a location trail of a first mobile device that is associated with the first person; providing identifiers of at least one wireless communication device to which the first mobile device could communicate; providing measurements on signals from the wireless communication devices; providing one or more maps marked with locations of the wireless communication device; determining the mobile device's location based upon the identifiers, the measurements and the maps; adding information about the location to a location trail associated with the mobile device that is associated with the first person; comparing, based upon the map marked with the wireless communication device, the location trail of the mobile device that is associated with the first person against a location trail of a second mobile device associated with a second person to determine if the other the mobile devices of the first person and the second person have been on approximately the same building floor at approximately the same time; whereby persons on the same floor with the person can be identified and separated from persons on other floors. In another embodiment, the method further includes the step of determining if the mobile device has been in the same room at approximately the same time with other mobile devices. The measurements on the signals from the wireless communication devices may include one or more of the following measurement types: received signal strength; distance from the wireless communication device to the mobile device; and propagation delay of the signal from the wireless communication device to the mobile device.

In another embodiment, there is presented a method of mapping the location of a mobile device associated with a person within an environment comprising: obtaining data needed to calculate time-bound location trails; sending the data to a map server, wherein the map server has a map of the environment that includes the location of wireless communication devices located within the environment; determining the mobile device's location based upon the data and the map; and storing the location in a location trail stored in a database, wherein the location trail is associated with the mobile device. In another embodiment, the method further includes the step of determining if the mobile device has been in the same room at approximately the same time with other mobile devices. The comparison step could be performed for the purpose of contact tracing.

Also presented is a method of contact tracing a person associated with a mobile device comprising: sending a location trail associated with the mobile device to an infected repository server; calculating an infectivity window of when an infected person can transmit a specific disease to another person; storing the location trail bounded by the infectivity window in a database; sending the location trail bounded by the infectivity window from the infected repository server to other mobile devices associated with other people; comparing the location trail bounded by the infectivity window to other location trails stored on the other mobile devices; and determining whether the other people have been exposed to the disease based upon the comparison.

Also presented is a method of contact tracing a person associated with a mobile device comprising: sending a location trail associated with the mobile device to a repository server; calculating a meeting window of when said person has met or been in contact with another person; storing the location trail bounded by the meeting window in a database; sending the location trail bounded by the meeting window from the repository server to other mobile devices associated with other people; comparing the location trail bounded by the meeting window to other location trails stored on the other mobile devices; and determining whether said person has met or been in contact with other people disease based upon the comparison.

In another embodiment, there is presented a distributed method of mapping the location of a mobile device associated with a person within an environment comprising: obtaining data needed to calculate time-bound location trails; sending the data to a map server, wherein the map server has a map of the environment that includes the location of wireless communication devices located within the environment; sending the map to the mobile device; determining the mobile device's location based upon the data and the map; and storing the location in a location trail stored in a database stored on the mobile device, wherein the location trail is associated with the mobile device.

In yet another embodiment, there is presented a method of contact tracing a person associated with a mobile device, wherein the person is infected or potentially infected with a specific disease, comprising: sending a location trail associated with the mobile device to an infected repository server; calculating a infectivity window of when an infected person can transmit the specific disease to another person; storing the location trail bounded by the infectivity window in a database; requesting and receiving the location trail bounded by the infectivity window, wherein the request is made by a device associated with a person other than the infected or potentially infected person; comparing the location trail bounded by the infectivity window to another location trail stored on the device requesting the location trail bounded by the infectivity window; and determining whether the other person has been exposed to the disease based upon the comparison.

In accordance with the present invention, the mapped environment can be an indoor environment, an outdoor environment or an environment comprising indoor and outdoor sections. The present invention is particularly useful for environments that include a multi-story building.

In each of the above embodiments, as well as other embodiments, the measurements of the signals received from the communication devices could be of the following types: received signal strength; distance from the wireless communication device to the first mobile device; and propagation delay of the signal from the wireless communication device to the first mobile device. In each of the above embodiments, as well as other embodiments, the wireless communication device could conform to one or a combination of the following standards: Wi-Fi; Bluetooth; LTE; UHF; GMRS; PMR446; LoRaWan; NB-IoT (Narrowband IoT); Sigfox; IEEE 802.11ah and IEEE 802.15.4z.

In each of the above embodiments, as well as other embodiments, the data used to calculate locations and location trails could include Global Positioning System (GPS) coordinates, identifiers of Wi-Fi access points within communication range, received signal strength of each Wi-Fi access point, and time stamp of each datum. For example, the received signal strength from the Wi-Fi access points could be converted to estimated distance measurements to the Wi-Fi access points through use of standard artificial intelligence and machine learning algorithms. Preferably, mobile device's location is determined using received signal strengths of Wi-Fi access points. In some embodiments, Bluetooth beacons could augment or replace the Wi-Fi access points. In other embodiments, inertial measurement units in the mobile devices can be used to augment or supersede measurements of received signal strength for purposes of calculating the location of the mobile device. Preferably, the inertial measurement units are military-grade or otherwise have high precision.

In each of the above embodiments, as well as other embodiments, the location of a mobile device can also be calculated based in part on propagation delays of wireless signals from the wireless communication devices. In certain embodiments, the propagation delays are estimated through modeling of the paths from the map server to the wireless communication device and from the wireless communication device to the mobile device. In certain embodiments, the location can be calculated based upon the Basic Service Set Identifier (BSSID) or the Extended Service Set Identifier (ESSID) in conjunction with the received signal strength.

As used in this application, a mobile device is a portable computing device, such as: a tablet computer; a laptop computer; a smart phone; a smart wireless watch; and a smart wireless badge.

The maps used in the present invention can be static maps or interactive maps. Preferably, the maps are interactive maps. An interactive map is a map, diagram or floor plan that includes hotspots. A hotspot is a location on the map that responds when the mouse hovers over or clicks on the hotspot. Typically, interactive maps allow zooming in and out, panning around, rotation, identifying specific features on the map, querying underlying data, generating reports and other means of using or visualizing selected information in the map. Preferably, the maps of the present invention have one or more of the following features: (i) multiple layers of elements (eg. store layer, facilities layer, etc.); (ii) each map element is a data object with properties and functionalities; (iii) each map element is searchable; (iv) routing is calculated for every origin element to every destination element; (v) data can be associated to every point (represented by coordinates) on the map; (vi) the map can be zoomed, panned, rotated; (vii) the map has proportional scale to earth such that time of travel can be computed from origin to destination; and (viii) separate floors on the map can be connected via vertical pathways like stairs and elevators. Interactive maps can be created using commercial software tools such as Mapwize Studio® (Contexeo SAS, Lille, France), Mapme™ (Mapme, Ra'anana, Israel), and MapsAlive™ (Avantlogic Corporation, Southwest Harbor, Me., USA).

Accordingly, several possible advantages of one or more aspects are as follows: to make going back to work, dining at a restaurant, etc. safer; to enable each person, employers, businesses, and public health authorities to determine more accurately whether people have been in contact inside of a building; to use existing equipment that most people already carry; and to use existing infrastructure like Wi-Fi access points. Not all of these advantages may be present in each embodiment of the invention. Other advantages of one or more aspects will be apparent from a consideration of the drawings and ensuing description.

### BRIEF DESCRIPTION OF THE DRAWINGS

The following detailed description of exemplary embodiments of the subject disclosure will be better understood when read in conjunction with the appended drawings. For the purpose of illustrating the present disclosure, exemplary embodiments are shown in the drawings. It should be understood, however, that the subject application is not limited to the precise arrangements and examples shown.

FIG. 1 is a diagram of a building with three floors, each with a person and Wi-Fi access points.

FIG. 2 is a flowchart of how mobile devices send their location data to map servers to get their positions inside of buildings.

FIG. 3 is a push-model flowchart of how contact tracing is done with location trails containing positions in buildings.

FIG. 4 is a flowchart of how mobile devices calculate their own positions inside buildings by utilizing maps that map servers send to them.

FIG. 5 is a pull-model flowchart of how contact tracing is done with location trails containing positions in buildings.

### DETAILED DESCRIPTION

Before describing the present invention in detail, it is to be understood that the invention is not limited to specific contact tracing systems or to contact tracing systems generally. It is also to be understood that the terminology used herein is for the purpose of describing particular embodiments only and is not intended to be limiting.

In addition, as used in this specification and the appended claims, the singular article forms “a,” “an,” and “the” include both singular and plural referents unless the context clearly dictates otherwise. Thus, for example, reference to “a mobile device” includes a single mobile device as well as a plurality of mobile devices, reference to “a system” refers to a single system as well as a plurality of systems, and the like. The term “mobile device,” for purposes of the claimed invention, means any portable computing device, including smart phones, tablets, personal digital assistants, laptop computers, smart badges and smart watches. Typically, mobile devices have the ability to transmit and receive information (e.g., via Wi-Fi, Bluetooth, cellular networks, near field communication such as UHF). Some mobile devices are wearable devices or implantable devices.

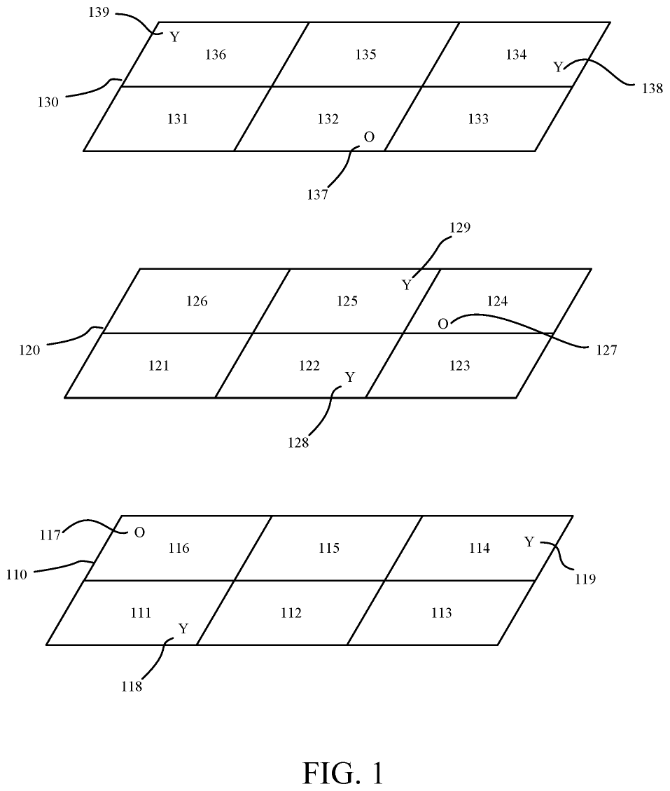

An exemplary system is shown in FIGS. 1-5.

FIG. 1 is a diagram of floors 110, 120 and 130 of a building. The walls of the building are not shown. Each floor has six rooms. Floor 110 has rooms 111 through 116, floor 120 has rooms 121 through 126, and floor 130 has rooms 131 through 136. The persons 117, 127, and 137 each occupy a separate floor. Each person has his or her own mobile device that he or she carries. Two Wi-Fi access points are on each floor. Floor 110 has Wi-Fi access points 118 and 119, floor 120 has Wi-Fi access points 128 and 129, and floor 130 has Wi-Fi access points 138 and 139.

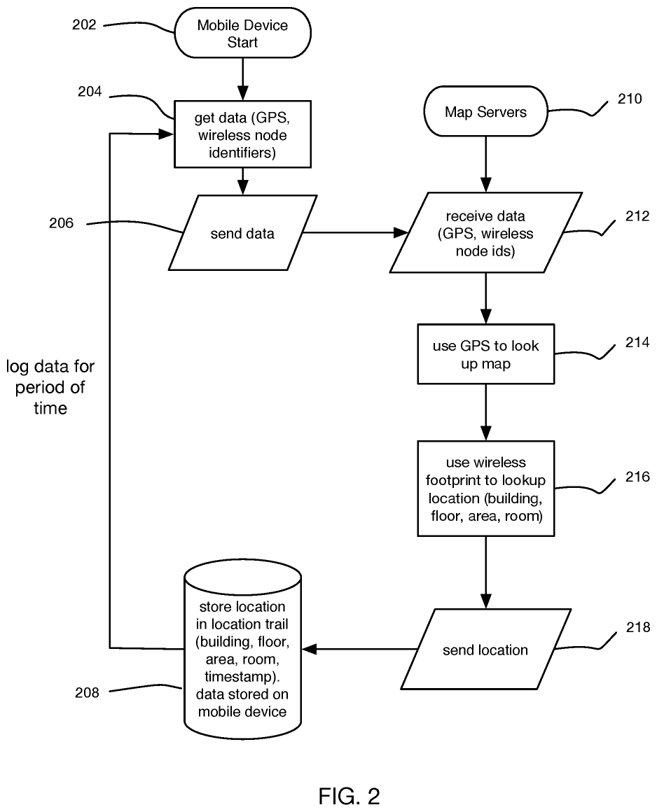

FIG. 2 is a flowchart of a method for the calculation of the location of the mobile devices. In step 204, the mobile device 202 gets data such as its Global Positioning System coordinates, the identifiers of Wi-Fi access points within communication range, the received signal strength of each Wi-Fi access point, and the time stamp of each datum. In step 206, the mobile device 202 then sends all the collected data to the map server 210. The data may be sent to an on-site or off-site server through the internet or by some other means. The data may be transmitted synchronously or asynchronously; moreover, the data may be transmitted at some regular time interval (i.e., scheduled) or upon user request (i.e., on-demand).

In step 212, the map server 210 receives the data from the mobile device. In step 214, the map server 210 first identifies through the GPS coordinates which building or section of a large building that the mobile device 202 is located in and then looks up the map corresponding to the building, building section or other indoor environment. An alternative approach to determining the location of the mobile device 202 is GSM (Global System for Mobile Communications) localization, which uses multilateration of radio signals between cell towers of a network (based on the mobile devices' signal strength to nearby antennas. Some methods of determining outdoor location include using a combination of GPS coordinates and dead reckoning sensors; for example, automotive navigation systems typically use dead reckoning. See, e.g., U.S. Pat. No. 5,583,776. In an indoor environment, Wi-Fi data can be used to replace or supplement GPS and/or GSM data to determine the location of a mobile device. Alternatives to GPS include GLONASS (Global Navigation Satellite System) or other variants of GNSS. Hybrid position systems (which combine several different location approaches, such as WiMAX, WiFi, LTE, IP addresses and network environment data) can also be used to ascertain the map coordinates of a mobile device. Next, the map server determines the location of the mobile device inside the building by calculating the position where the received signal strengths of the Wi-Fi access points would approximately correlate to the known measurements. For example, the mobile device 202 of person 117 is within communications range of Wi-Fi access points 118, 119, 128, and 129 and therefore measures their received signal strength. The measurements are sent to the map server 210, which calculates that person 117 is in room 116.

Received signal strength is a measurement of the power level received by sensor on the mobile device. Because radio waves propagate according to the inverse-square law, distance can be approximated based on the relationship between transmitted and received signal strength, as long as no other errors contribute to faulty results or such errors are taken into account. For example, accuracy is significantly impacted by reflection and absorption from walls, and non-stationary objects such as doors, furniture, and people can affect the signal strength in dynamic, unpredictable ways. Data regarding received signal strength from Wi-Fi access points can be supplemented by data relating to received signal strength of Bluetooth beacons, Ultra-Wideband beacons or other sources of signals. Methods of using received signal strength to determine indoor location include RSSI-based fingerprinting, angle-of-arrival (AOM) based approaches (e.g., using the MUSIC (Multiple Signal Classification) algorithm) for triangulation or multilateration, and time-of-flight (Tot) localization approaches. See, e.g., U.S. Pat. Nos. 7,411,549; 7,856,209; 9,185,528; and 9,554,247. Triangulation methods typically uses Friis' formula to relate signal loss to distance from a source. However, given that radio wave signals can reflect and cause interference, one must take into account multipath propagation to accurately calculate location.

Fingerprint methods rely on comparing current measurements of the signal strength fingerprint by a mobile device with a database of fingerprints mapped to specific locations. The fingerprint database may be populated using empirical data obtained through an extensive site survey, using an RF predictive model, or a combination of empirical data and a predictive model. The fingerprint database may be crowd-sourced. Artificial intelligence methods can be used to improve the accuracy of fingerprint methods; for example, a random forest ensemble learning method can be used for classification and regression analysis in constructing fingerprint maps. See S. Lee et al., “Random Forest and WiFi Fingerprint-Based Indoor Location Recognition System Using Smart Watch,” Human-Centric Computing and Information Sciences 9, 6 (2019), https://doi.org/10.1186/s13673-019-0168-7 (accessed Jun. 2, 2020). As used in the present application, the term “wireless footprint” is synonymous with the term “Wi-Fi fingerprint,” which is more commonly used in the literature.

Likewise, the mobile device 202 of person 127 is within communications range of Wi-Fi access points 118, 119, 128, 129, and 138 and sends the measurements of the received signal strength from those access points to the map server, which calculates that the mobile device 202 of person 127 is in room 124. Lastly, the mobile device 202 of person 137 is within communications range of Wi-Fi access points 128, 129, 138, and 139 and sends the measurements of the received signal strength from those access points to the map server, which calculates that the mobile device of person 137 is in room 132.

In step 216, the wireless footprint (aka Wi-Fi fingerprint) is used to determine the location of the mobile device 202. In step 218, the location of the mobile device 202 is transmitted to database 208, which stores the location in its location trail. The database 208 can be located on the mobile device 202 or on a remote server. The advantage of storing the data on the mobile device is enhanced privacy protection albeit with the disadvantage of more limited storage capacity. If stored on a remote server or other device, the data can be encrypted to protect privacy. Then the process loops back to step 204.

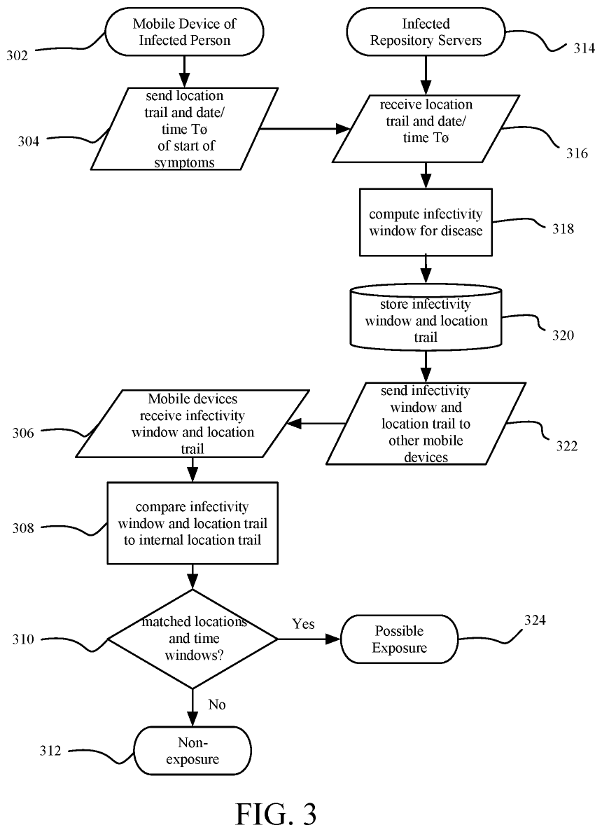

FIG. 3 shows the push-model flowchart of how to perform contact tracing of people within an environment such as a building or other defined area. In the depicted process, each person who is infected or potentially infected with a disease possesses an associated mobile device 302, which can be used for contact tracing. In step 304, the mobile device associated with the infected person or person of interest sends its location trail and the date/time To, either through the internet or by some other means, to infected repository server 314. The date/time To is either the time when the infected or potentially infected person began to experience symptoms or the time at which it is desired to begin tracking the person of interest.

An infected repository server is a server (i.e., a computer or other device) that hosts a repository (i.e., database) of information (such as the location trails) about infected or potentially infected individuals. A repository server is a server (i.e., a computer or other device) that hosts a repository (i.e., database) of information (such as the location trails) about individuals who are being tracked. Infected or potentially infected individuals can submit information such as their location trails and the date or time when they first showed symptoms of infection to such infected repository server. Similarly, information can be submitted about individuals who are being tracked to a repository server.

The infected repository server may also store information concerning a disease or diseases, such as the incubation period of such disease or diseases and the infectivity window. The incubation period is the time period between exposure to an infection and the appearance of the first symptoms. The infectivity window is the period of time that the pathogen or infectious agent in question can survive in the air (e.g., in aerosol form) or on a surface and is capable of infecting an individual after such pathogen or infectious agent has been released into the air or deposited on a surface. Notably, an uninfected individual can be exposed and infected even if such individual has not been in the vicinity of or in the same room as an infected individual if the uninfected individual touches a contaminated surface or breathes in contaminated air during the infectivity window. For example, if a pathogen or infectious agent is able to survive and retain infectious activity on a surface for one hour after being deposited on such surface, then an uninfected individual could become infected if the individual touches the surface within one hour after an infected individual has touched such surface.

In step 316, the infected repository server receives the location trail and the date/time To when the infected or potentially infected person began to experience symptoms; and in step 318, the infectivity window of when the infected person can transmit the disease to another person is computed. Alternatively, in step 316, the repository server receives the location trail and the date/time To that it is desired to begin tracking a particular person; and in step 318, the meeting window is calculated or retrieved. The meeting window is the minimum period of time that two individuals would be in the vicinity of each other as to constitute being in contact with each other or having met. The location trail bounded by the infectivity window or the meeting window is then stored in database 320. In step 322, the location trail bounded by the infectivity window is sent, through the internet or by some other means, to the other mobile devices in the network. These mobile devices accept the infectivity window-bounded location trail of the potentially infected person in step 306. In step 308, the mobile devices compare their own location trails against the infectivity window-bounded location trail of the potentially infected person. In step 310, the mobile device determines whether there has been an exposure based on the comparison of location trails in light of the incubation period and the infectivity window: if there are matches in the location trails, then the mobile device reaches conclusion 324 (i.e., the people associated with the respective mobile devices have been exposed to the infected person); if there are no matches in the location trails, then the mobile device reaches conclusion 312 (i.e., the people associated with the mobile devices have not been exposed to the infected person).

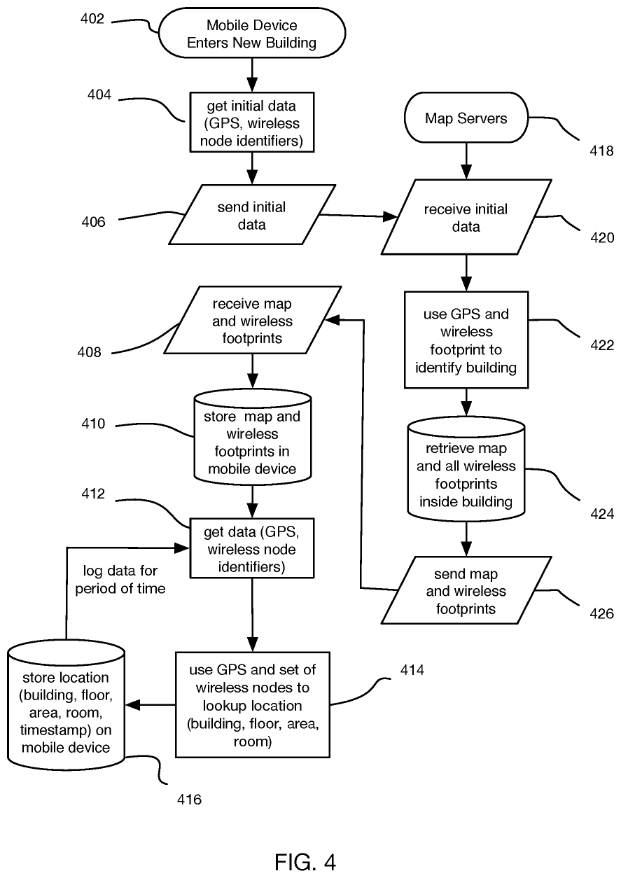

In another embodiment, FIG. 4 is a flowchart of a distributed method for the calculation of the location of the mobile devices. The process starts in step 402, in which a person carrying a mobile device enters a new building. In step 404, the mobile device gets initial data such as its Global Positioning System coordinates, the identifiers of Wi-Fi access points within communication range, the received signal strength of each Wi-Fi access point, and the time stamp of each datum. In step 406, the mobile device sends all the collected initial data, through the internet or by some other means, to the map server 418.

In step 420, the map server 418 receives the initial data from the mobile device. By analyzing the initial data, the map server, in step 422, identifies the building or section of a large building in which the mobile device is, using GPS coordinates and wireless footprint data. Next, the map server retrieves the respective map of the identified building from database 424. In step 426, the map and wireless footprint data are sent, through the internet or by some other process, to the mobile device that sent the initial data to the map server 418.

In step 408, the mobile device receives the map and wireless footprints from the map server 418. The map and wireless footprint data are stored in database 410, which is stored in the memory of the mobile device. For the rest of the time that the mobile device is located in the building associated with the map, it will perform the following steps in a repeating loop: (1) get data such as GPS location, Wi-Fi access point identifiers, etc. (step 412); (2) calculate the location of the mobile device inside of the building, including the room and floor (step 414); and (3) store the location in the location trail of the mobile device (database 416).

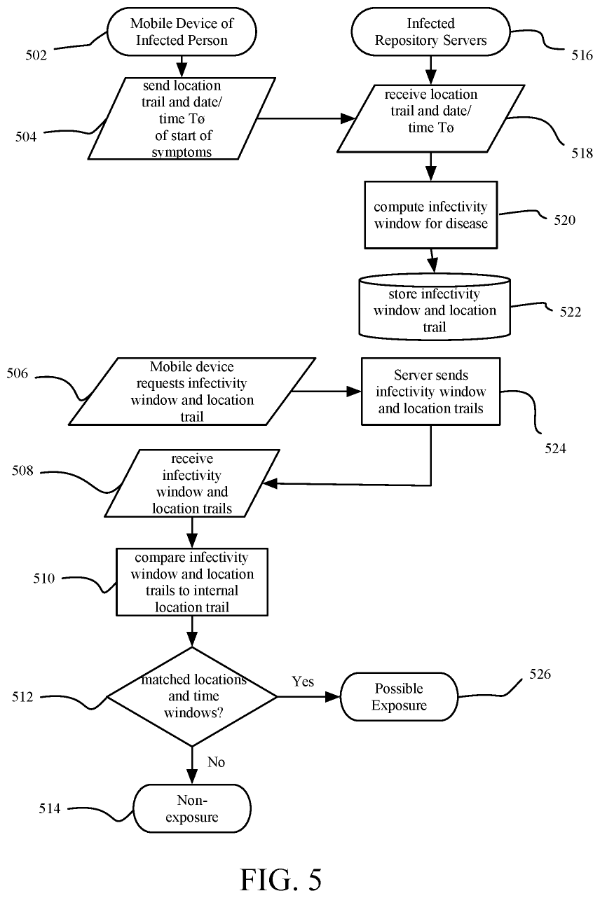

In yet another embodiment, FIG. 5 shows the pull-model flowchart of how to perform contact tracing of people inside of a building. In the depicted process, the mobile device 502 associated with (i) a person who is infected or potentially infected with a disease or (ii) a person who is being tracked is used for contact tracing. In step 504, the mobile device 502 of (i) the infected or potentially infected person or (ii) the person being tracked sends its location trail and the date/time To, through the internet or by some other means, to the infected repository server 516. The date/time To either the time when the infected or potentially infected person began to experience symptoms or the time at which it is desired to begin tracking the person of interest. In step 518, the infected repository server 516 receives the location trail and the date/time To when the infected or potentially infected person began to experience symptoms; in step 520, the infectivity window of when the infected person can transmit the disease to another person is computed. Alternatively, in step 518, the repository server 516 receives the location trail and the date/time To that it is desired to begin tracking a particular person; and in step 520, the meeting window is calculated or retrieved. The location trail bounded by the infectivity window or meeting window is stored in database 522.

In step 506, on a pre-determined schedule, each mobile device (that is associated with a different person than the infected people) requests that the infected repository servers send the infectivity window-bound location trails of the infected people. In step 524, the infected repository server 516 retrieves and sends the latest time-bounded location trails of the infected people to the mobile device that sent the request. The mobile device accepts the infectivity window-bound location trails of the infected people in step 508. In step 510, the mobile device compares its own location trail against the infectivity window-bound location trails of the infected people. In step 512, the mobile device determines whether there has been an exposure based on the comparison of location trails in light of the incubation period and the infectivity window: if there are matches in the location trails, then the mobile device reaches conclusion 526 (i.e., the person associated with the respective mobile device has been exposed to the infected person); if there are no matches in the location trails, then the mobile device reaches conclusion 514 (i.e., the person associated with the mobile device has not been exposed to the infected person).

In another embodiment of the invention, the propagation delays of wireless signals can used to calculate the location of mobile devices. Stationary wireless communication devices within the building transmit time stamps to the mobile devices, thereby enabling the mobile devices to calculate the propagation delay of the wireless signal through air. The distance from the wireless communication device to the mobile device is calculated from the propagation delay. The mobile device sends either the propagation delay or the calculated distance to the map server, which then calculates the position where the mobile device would correlate to the measurements.

Thus, a number of conclusions, ramifications, and scoping issues are apparent from the above disclosure. For example, contact tracing in the context of a map provides more insightful results for people living or working in multistory buildings in urban areas with high density. In such areas, highly infectious airborne diseases can spread more easily and quickly through the population. Therefore, contact tracing tools need to be faster and more accurate to control an outbreak of the disease. In addition, the increasing numbers of Wi-Fi access points and other radio transceivers facilitates the localization of people with their mobile devices inside of a building.

Furthermore, the more detailed localization of people inside of a building has the following benefits: people who are close together spatially but in separate floors or rooms will not be falsely assumed to have been exposed to each other; approximate estimates can be made of the time duration that people have been exposed to each other so that better assessments can be made of the chance of infection; and more sophisticated programs can more easily analyze the contact trace data because of its electronic nature.

Additional embodiments of the invention can include one or more of the following features: Bluetooth beacons can be used to augment or replace the Wi-Fi access points for the purpose of localization; the received signal strength from the Wi-Fi access points can be converted to estimated distance measurements to the Wi-Fi access points through the use of standard artificial intelligence and machine learning algorithms; propagation delays of the wireless signals are estimated through modeling of the delay paths from map server to wireless communication device and wireless communication device to mobile device; inertial measurement units (preferably military-grade or otherwise high precision) in the mobile devices are used to augment or supersede the measurements of the wireless signal for the purposes of mapping users; models of the time decay of virus particles in the air can improve estimates of the likelihood of infection; models of the airflow in a room or between rooms can improve estimates of the likelihood of infection.

For the sake of brevity, all publications, including patent applications, patents, and other citations mentioned herein, are specifically and individually incorporated by reference in their entirety. Citation of any such publication, however, shall not be construed as an admission that it is prior art to the present invention.

While this invention has been particularly shown and described with references to preferred embodiments thereof, it will be understood by those skilled in the art that various changes in form and details can be made therein without departing from the scope of the invention encompassed by the embodiments. Further, all embodiments included herein are given solely for the purpose of illustration and are not to be construed as limitations of the present invention, as many variations thereof are possible without departing from the spirit and scope of the invention. Thus, the scope of the embodiments should be determined by the appended claims and their legal equivalents, rather than by the examples given.

What is claimed is:

1. A mapping system comprising:
a first mobile device that is associated with a first person;
a wireless communication device which can communicate to said first mobile device, wherein said communication device sends identifying information about said communication device to said first mobile device and wherein said first mobile device receives measurements on signals received from said wireless communication device;
a map stored on said first mobile device, wherein said map is marked with location of said wireless communication device;
whereby a first location trail is calculated based upon said map, said identifying information and said measurements, and wherein said location trail is stored on said first mobile device;
a second mobile device associated with a second person, wherein said second mobile device receives said first location trail and compares said first location trail with a second location trail associated with said second person in order to determine whether said first mobile device has been on the same building floor at approximately the same time as said second mobile device.

2. The system of claim 1, wherein said second mobile device determines if said first mobile device has been in the same room at approximately the same time as said second mobile device.

3. The system of claim 1, wherein said measurements comprise at least one of the following measurement types:
received signal strength;
distance from said wireless communication device to said first mobile device; and
propagation delay of said signal from said wireless communication device to said first mobile device.

4. The system of claim 1, wherein said wireless communication device conforms to one or a combination of the following standards:
Wi-Fi;
Bluetooth;
LTE;
UHF;
GMRS;
PMR446;
LoRaWan;
NB-IoT (Narrowband IoT);
Sigfox;
IEEE 802.11ah and
IEEE 802.15.4z.

5. The system of claim 1, wherein said first mobile device is selected from the group of devices consisting of:
a tablet computer;
a laptop computer;
a smart phone;
a smart wireless watch; and
a smart wireless badge.

6. A system for mapping the location of a mobile device associated with a person within an environment comprising:
a map server which stores a map of said environment that includes the location of wireless communication devices located within said environment;
wherein said mobile device obtains data needed to calculate time-bound location trails and send said data to said map server;
wherein said map server determines said mobile device's location based upon said data and said map, and stores said location in a location trail stored in a database, wherein said location trail is associated with said mobile device.

7. The system of claim 6, wherein said map is an interactive map.

8. The system of claim 6, wherein said map server also compares said location trail associated with said mobile device with other location trails associated with other mobile devices.

9. The system of claim 8, wherein said comparison step is performed in order to perform contact tracing of said person.

10. The system of claim 6, wherein said data includes Global Positioning System coordinates, identifiers of Wi-Fi access points within communication range, received signal strength of each Wi-Fi access point, and time stamp of each datum.

11. The system of claim 10, wherein said received signal strength from said Wi-Fi access points is converted to estimated distance measurements to said Wi-Fi access points through use of standard artificial intelligence and machine learning algorithms.

12. The system of claim 10, wherein Bluetooth beacons augment or replace said Wi-Fi access points.

13. The system of claim 10, wherein inertial measurement units in said first mobile device and said other mobile devices are used to augment or supersede measurements of received signal strength for purposes of calculating the location of said mobile device.

14. The system of claim 6, wherein said data is sent to said map server through the internet.

15. The system of claim 6, wherein said mobile device's location is determined using received signal strengths of Wi-Fi access points.

16. The system of claim 6, wherein said environment is an indoor environment.

17. The system of claim 6, wherein said environment comprises indoor and outdoor sections.

18. The system of claim 6, wherein said environment is a multi-story building.

19. The system of claim 6, wherein said location is calculated based in part on propagation delays of wireless signals from said wireless communication devices.

20. The system of claim 19, wherein said propagation delays are estimated through modeling of the paths from said map server to said wireless communication device and from said wireless communication device to said mobile device.
